(19) United States
(12) Patent Application Publication
(10) Pub. No.: US 2004/0053270 A1
(43) Pub. Date: Mar. 18, 2004

(54) RNA binding compounds and uses thereof

(76) Inventors: Alastair Iain Hamilton Murchie, Abington (GB); Georg Fredrich Lentzen, Abington (GB)

Correspondence Address: PALMER & DODGE, LLP, KATHLEEN M. WILLIAMS, 111 HUNTINGTON AVENUE, BOSTON, MA, 02199, US

(21) Appl. No.: 10/365,826

(22) Filed: Feb. 13, 2003

Related U.S. Application Data
(63) Continuation of application No. PCT/GB01/03611, filed on Aug. 13, 2001.

(30) Foreign Application Priority Data
Aug. 15, 2000 (GB) 0020083.2

(51) Int. Cl. C12Q 1/68; C07H 21/02; C07H 21/04; C07D 413/02

(52) U.S. Cl. 435/6; 536/23.1; 544/369

(57) ABSTRACT

A method for determining whether a test compound binds to a large RNA target, comprising the steps of: (a) contacting the test compound with a pair of indicator molecules comprising (i) a fluorescent oxazolidinone or aminoglycoside reporter molecule and (ii) the large RNA target; and (b) measuring the fluorescence of reporter molecule in the presence of the test compound and comparing this value to the fluorescence of the reporter in the absence of the test compound. It has been found that large RNA molecule are advantageous in this type of binding assay.

### RELATED APPLICATIONS

This application is a continuation of PCT/GB01/03611, filed Aug. 13, 2001, which claims the benefit of GB Application No. 0020083.2, filed Aug. 15, 2000, the entirety of which is hereby incorporated by reference.

### TECHNICAL FIELD

This invention is in the field of assays for compounds which interact with RNA. More particularly, it is in concerned with the identification of improved antibiotics.

### BACKGROUND ART

In most biological systems, the maturation, transport, stability and expression of RNA is closely regulated by the interactions between highly conserved regulatory RNA sequences and proteins. In many circumstances it is desirable to develop drugs that bind RNA at sites of regulatory protein binding and act as competitive inhibitors of the RNA-protein interaction. These types of drugs have potential application in a wide range of diseases including viral, bacterial and fungal infections and chronic diseases such as cancer and autoimmune disease.

To identify RNA-binding compounds, methods involving fluorescence have been reported.

Reference 1 discloses methods in which a compound and a RNA are labelled with fluorescent donor and acceptor groups. Interaction between the compound and RNA allows the donor and acceptor groups to interact, resulting in a detectable change (quenching). Reference 2 discloses methods in which aminoglycosides are fluorescently labelled in order to monitor their binding to RNA molecules. Both references point towards reducing the size of the RNA towards a “minimal motif” for use in the assay.

### DISCLOSURE OF THE INVENTION

The invention is based on the finding that reducing the size of RNA does not necessarily give the best results. In particular, it has been found that small RNA molecules of the type disclosed in reference 2 may not bind antibiotics, whereas larger RNA molecules or complexes retain antibiotic-binding activity. This suggests that tertiary interactions are important for the formation of an antibiotic-RNA complex.

The invention provides a method for determining whether a test compound binds to a large RNA target, the method comprising the steps of:

(a) contacting the test compound with a pair of indicator molecules comprising (i) the large RNA target; and (ii) a fluorescent reporter molecule, wherein the reporter molecule is an oxazolidinone or an aminoglycoside; and

(b) measuring the fluorescence of reporter molecule in the presence of the test compound and comparing this value to the fluorescence of the reporter in the absence of the test compound.

In contrast to the methods disclosed in reference 1, the fluorescent group on the reporter does not interact with a fluorescent group on the RNA target.

The Large RNA Target

Contrary to the trends in the prior art, the method of the present invention utilises large RNA molecules.

The RNA target will comprise more than 250 nucleotides, typically more than 350 nucleotides, and often more than 500 nucleotides.

The RNA target will be able to fold to adopt a complex structure. In particular, it may contain up to N base pairs, where N=(total number of nucleotides in RNA)/2 e.g. at least 0.5N base pairs, preferably at least 0.7N base pairs, more preferably at least 0.8N base pairs, and most preferably 0.9N base pairs.

The RNA target will preferably comprise at least one of the following secondary structure motifs (i.e. at least one discontinuity in normal base-paired double-helical RNA): bulged (non base-paired) and extra-helical bases; internal loops; helical junctions; G-quartets; and pseudoknots. The RNA target may comprise inter-domain (inter-helical or inter-secondary structure) molecular interactions, which may include the ligand binding site.

It may also comprise protein in the form of a RNP.

Examples of preferred RNA targets include viral genomes, RNAs comprising an IRES, nucleoprotein complexes (e.g. RNPs, SNRPs), mRNAs, rRNAs (e.g. complete rRNAs such as 23S rRNA, 16S rRNA etc.), ribosomal subunits, and whole ribosomes.

The RNA target may comprise one or more RNA strands, and it may comprise natural or synthetic RNA.

The Fluorescent Reporter p RNA-binding molecules typically contain functional groups (e.g. primary and secondary amine, hydroxyl, nitro, carbonyl etc.) which can be derivatised using fluorescent dyes to prepare reporters. Thus the reporter is prepared by labeling a compound which (a) binds to RNA e.g. an antibiotic and (b) has an atom to which a fluorophore can be covalently attached. The labelled compound can undergo a measurable change when it binds to RNA, and is thus useful in binding and displacement assays.

The fluorescent reporter used in the methods of the invention is an oxazolidinone or an aminoglycoside, to which is attached a suitable fluorophore. The fluorophore may be any fluorophore which does not interfere with the ability of the oxazolidinone or aminoglycoside to interact with the RNA target. A preferred fluorophore is TAMRA.

Other useful fluorophores include, but are not limited to: Texas Red™ (TR), Lissamine™ rhodamine B, Oregon Green™ 488 (2′,7′-difluorofluorescein), carboxyrhodol and carboxyrhodamine, Oregon Green™ 500, 6-JOE (6-carboxy-4′,5′-dichloro-2′,7′-dimethyoxyfluorescein, eosin F3S (6-carboxymethylthio-2′,4′,5′,7′-tetrabromotrifluorofluorescein), cascade blue™ (CB), aminomethylcoumarin (AMC), pyrenes, dansyl chloride (5-dimethylaminonaphthalene-1-sulfonyl chloride) and other naphthalenes, PyMPO, ITC (1-(3-isothiocyanatophenyl)-4-(5-(4-methoxyphenyl)oxazol-2-yl)pyridinium bromide).

Oxazolidinones are antibiotics which contain a substituted 2-oxazolidinone group:

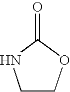

It will be appreciated that various substitutions can be made in this ring structure without affecting antibiotic activity. In particular, the NH group can be substituted with various groups (typically aromatic) and the CH₂ adjacent to the —O— group can be substituted (e.g. with —CH₂NH—COCH₃) [see, for example, references 3, 4, 5, 6, 7, 8, 9 etc.]

According to a preferred embodiment, the oxazolidinone employed in the present invention has the formula

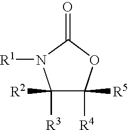

wherein R¹ to R⁵ are each independently selected from a σ bonding substituent.

Preferably R¹ to R⁵ are independently selected from halogen, hydrogen, C₁₋₁₂ alkyl, C₂₋₁₂ alkenyl, C₃₋₁₂ aryl, C₄₋₁₈ aralkyl.

Preferably R¹ is an optionally substituted C₃₋₁₂ aryl group. More preferably R¹ is a substituted phenyl group. More preferably, R¹ is a phenyl group substituted with 1 to 3 substituents, preferably 1 or 2 substituents, selected from halogen and heterocyclic groups. The heterocyclic groups may be further substituted.

Preferably R², R³ and R⁴ are hydrogen.

Preferably R⁴ and R⁵ are independently selected from hydrogen and substituted C₁₋₁₂ alkyl. More preferably R⁴ is hydrogen and R⁵ is substituted C₁₋₁₂ alkyl. More preferably R⁵ is an acetamidomethyl group. Oxazolidinone antibiotics are reviewed in References 25 and 26.

Oxazolidinones may have aldehyde groups which can be labelled with a fluorophore. Labelled oxazolidinones are preferred reporters for use in the invention. The label is preferably TAMRA, which may be attached as shown in FIG. 1. Preferred large RNA targets for use with oxazolidinone reporters comprise the E-site RNA (FIG. 3) or the L1 (e.g. a rRNA).

Preferred oxazolidinones are those which interact at the ribosome E (‘exit’) site [10].

A preferred oxazolidinone is “DuPont 721”, which binds to the 23S subunit of the ribosome at the nucleotides and ribosomal proteins that constitute the E site for tRNA binding, including nucleotides 2113, 2114, 2118, 2119 and 2153. There are also interactions with nucleotide 864 in the 16S subunit [3].

As used herein, the term “alkyl” means an optionally substituted branched or unbranched, cyclic or acyclic, hydrocarbyl radical. Where acyclic, the alkyl group is preferably a C₁₋₁₂, more preferably C₁₋₄ chain. Where cyclic, the alkyl group is preferably a C₃₋₁₂, more preferably C₅₋₁₀ and more preferably comprises a C₅, C₆ or C₇ ring.

As used herein, the term “alkenyl” means an optionally substituted branched or unbranched, cyclic or acyclic, hydrocarbyl radical comprising at least one double bond. Where acyclic, the alkenyl group is preferably a C₁₋₁₂, more preferably C₁₋₄ chain. Where cyclic, the alkenyl group is preferably a C₃₋₁₂, more preferably C₅₋₁₀ and more preferably comprises a C₅, C₆ or C₇ ring.

As used herein, the term “aryl” means an optionally substituted C₃₋₁₂ aromatic group, such as phenyl or naphthyl, or a heteroaromatic group containing one or more, preferably one, heteroatom, such as pyridyl, pyrrolyl, furanyl, thienyl.

As used herein, the term “aralkyl” means an optionally substituted branched or unbranched cyclic or acylic C₄₋₁₈ group comprising an alkyl group and an aryl group (for example, benzyl). An aralkyl group may be bonded via the alkyl or aryl group.

The alkyl, alkenyl, aryl, aralkyl and heterocyclic groups may be substituted or unsubstituted. Where substituted, there are preferably one to three substituents, more preferably one substituent. Substituents may include halogen atoms and halogen containing groups such as haloalkyl (e.g. trifluoromethyl); oxygen containing groups such as alcohols (e.g. hydroxy, hydroxyalkyl, aryl(hydroxy)alkyl), ethers (e.g. alkoxy, alkoxyalkyl, aryloxyalkyl), aldehydes (e.g. carboxaldehyde), ketones (e.g. alkylcarbonyl, alkylcarbonylalkyl, arylcarbonyl, arylalkylcarbonyl, arylcarbonylalkyl), acids (e.g. carboxy, carboxyalkyl), acid derivatives such as esters (e.g. alkoxycarbonyl, alkoxycarbonylalkyl, alkycarbonylyoxy, alkycarbonylyoxyalkyl) and amides (e.g. aminocarbonyl, mono- or dialkylaminocarbonyl, aminocarbonylalkyl, mono- or dialkylaminocarbonylalkyl, arylaminocarbonyl); and carbamates (e.g. alkoxycarbonylamino, aryloxycarbonylamino, aminocarbonyloxy, mono- or dialkylaminocarbonyloxy, arylaminocarbonyloxy), and ureas (e.g. mono- or dialkylaminocarbonylamino or arylaminocarbonylamino); nitrogen containing groups such as amines (e.g. amino, mono- or dialkylamino, aminoalkyl, mono- or dialkylaminoalkyl), azides, nitriles (e.g. cyano, cyanoalkyl), nitro; sulfur containing groups such as thiols, thioethers, sulfoxides, and sulfones (e.g. alkylthio, alkylsulfinyl, alkylsulfonyl, alkylthioalkyl, alkylsulfonylalkyl, alkylsulfonylalkyl, arylthio, arylsulfinyl, arylsulfonyl, arylthioalkyl, arylsulfinylalkyl, arylsulfonylalkyl). The alkyl, alkenyl, aryl and aralkyl groups may also be substituted with heterocyclic groups containing one or more, preferably one, heteroatom (e.g. thienyl, furanyl, pyrrolyl, imidazolyl, pyrazolyl, thiazolyl, isothiazolyl, oxazolyl, oxadiazolyl, thiadiazolyl, pyrrolidinyl, pyrrolinyl, imidazolidinyl, imidazolinyl, pyrazolidinyl, tetrahydrofuranyl, pyranyl, pyronyl, pyridyl, pyrazinyl, pyridazinyl, piperidyl, piperazinyl, morpholinyl, thianaphthyl, benzofuranyl, isobenzofuranyl, indolyl, oxyindolyl, isoindolyl, indazolyl, indolinyl, 7-azaindolyl, benzopyranyl, coumarinyl, isocoumarinyl, quinolinyl, isoquinolinyl, naphthridinyl, cinnolinyl, quinazolinyl, pyridopyridyl, benzoxazinyl, quinoxalinyl, chromenyl, chromanyl, isochromanyl, phthalazinyl and carbolinyl).

As used herein, the term “halogen” means a fluorine, chlorine, bromine or iodine radical, preferably a fluorine or chlorine radical. It will be appreciated that the compounds of the present invention may exist in a number of diastereomeric and enantiomeric forms. The present invention encompasses pure diastereomers and enantiomers as well as mixtures (including racemic mixtures) of diastereomers and enantiomers.

Aminoglycosides are broad-spectrum antibiotics that contain an aminodeoxysugar, an amino- or guanidino-substituted inositol ring, and one or more residues of other sugars. Preferably, the aminoglycosides contain one or more aminocyclitol rings (hexose nucleus), amino or guanidino-substituted inositol rings and may be attached to aminodeoxysugars or other sugars by glycosidic bonds. The structure of various aminoglycosides are shown in reference 11. Preferred classes and individual compounds are set out in the following table.

| | | | | | | in | | |
|---|---|---|---|---|---|---|---|---|
| | | | | Acting | Order | clinical | Vet | |
| Class | Antibiotic | Synonyms | Target | on | information | use | use | Ref. |
| 4,6-disubstituted | Amikacin | | A site | | Sigma | + | | J Infect Dis 1998 Jun; |
| deoxystreptamine | | | | | A3650 | | | 177(6): 1573-81 |
| core | | | | | | | | |
| Aminoglycoside | Apramycin | Nebramycin II | A site | | Sigma | − | + | EMBO J 1991 Oct; |
| | | | | | A2024 | | | 10(10): 3099-103 |
| 4,6-disubstituted | Kanamycin B | Bekanamycin | A site | | Sigma | | | Biochemistry 1998 Jan 13; |
| deoxystreptamine | | | | | B5264 | | | 37(2): 656-63 |
| core | | | | | | | | |
| Aminoglycoside | Bluensomycin | | ? | | ? | | | RNA 1998 Jan; 4(1): 112- |
| (streptomycin | | | | | | | | 23 |
| type) | | | | | | | | |
| 4,5-disubstituted | Butirosin A | | | | | | | |
| deoxystreptamine | | | | | | | | |
| core | | | | | | | | |
| Aminoglycoside | Fortimycin | | | | | | | |
| 4,6-disubstituted | Gl 8 | | | | Sigma | | | Biochemistry 1984 Mar |
| deoxystreptamine | | | | | G5013 | | | 27; 23(7): 1462.7 |
| core | | | | | | | | |
| 4,6-disubstituted | Gentamicin | Gentamycin | A site | | Sigma | + | | EMBO J 1998 Nov 16; I |
| deoxystreptamine | | | (L6) | | G1264 | | | 7(22): 6437-48 |
| core | | | | | (G6896) | | | |
| Aminoglycoside | Hygromycin B | | | | | | | Nature 1987 June 4-10; |
| | | | | | | | | 327(6121): 389-94 |
| Aminoglycoside | Istamycin | | A site | | | | | Mol Gen Genet 1984; |
| | | | | | | | | 197(1): 24-9 |
| 4,6-disubstituted | Kanamycin A | | A site | | Sigma | + | | J Mol Biol 1987 Feb 20; |
| deoxystreptamine | | | | | K0879 | | | 193(4): 661-71 |
| core | | | | | | | | |
| Aminoglycoside | Kasugamycin | | P site | pro | Sigma- | | | EMBO J 1991 Oct; |
| | | | | | Aldrich rare | | | 10(I0): 3099-103 |
| | | | | | chemicals | | | |
| | | | | | 78, 711-6 | | | |
| Aminoglycoside | Lividomycin A | | | | | | | |
| (Neomycin | | | | | | | | |
| group) | | | | | | | | |
| Aminoglycoside | Myomycin | | ? | | ? | | | EMBO J 1991 Oct; |
| | | | | | | | | 10(10): 3099-103 |
| Aminoglycoside | Neamin | | A site | | | | | EMBO J 1991 Oct; |
| (Neomycin | | | | | | | | 10(10): 3099-103 |
| group) | | | | | | | | |
| 4,5-disubstituted | Neomycin | Fradiomycin | A site | | Sigma | + | | J Mol Biol 1999 Feb 12; |
| deoxystreptamine | | | | | N1876 | | | 286(1): 33-43 |
| core | | | | | | | | |
| 4,6-disubstituted | Netilmicin | | ? | | Sigma | + | | J Antimicrob Chemother |
| deoxystreptamine | | | | | N0755 | | | 1984 Sep; 14(3): 231-41 |
| core | | | | | | | | |
| 4,5-disubstituted | Paromomycin | | A site | | Calbiochem | | | J Mol Biol 1998 Mar 27; |
| deoxystreptamine | | | | | 512731 | | | 277(2): 333-45 |
| core | | | | | | | | |
| 4,5-disubstituted | Ribostamycin | | A site | | Sigma | | | J Mol Biol 1998 Mar 27; |
| deoxystreptamine | | | | | R2255 | | | 277(2): 347-62 |
| core | | | | | | | | |
| Aminoglycoside | Sagamicin | | ? | | | | | J Antibiot (Tokyo) 1983 |
| (Kanamycin | | | | | | | | Feb; 36(2): 125.30 |
| group) | | | | | | | | |
| 4,6-disubstituted | Sisomycin | | ? | | Calbiochem | | | J Bacteriol 1992 Dcc; |
| deoxystreptamine | | | | | 567205 | | | 174(23): 7868-72 |
| core | | | | | | | | |
| Aminoglycoside | Sorbistin | | ? | | | | | J Antibiot (Tokyo) 1976 |
| | | | | | | | | Nov; 29(11): 1152-62 |
| Aminocylitol | Spectinomycin | | S5 | pro | Calbiochem | + | + | J Mol Biol 1997 Aug 29; |
| | | | binding | | 567570 | | | 271(4): 566-87 |
| | | | site | | | | | |
| Aminoglycoside | Streptomycin | Actinamine | A site, | | Caliochem | + | | J Mol Biol 1997 Oct 31; |
| (streptomycin | | | 915 | | 5711 | | | 273(3): 586-99 |
| type) | | | region, | | | | | |
| | | | S12, | | | | | |
| | | | S5?, | | | | | |
| | | | L11? | | | | | |
| 4,6-disubstituted | Tobramycin | Streptidine | A site | | Sigma | + | | Nature 1987, 327, 389- |
| deoxystreptamine | | | | | T4014 | | | 394 |
| core | | | | | | | | |
| Aminoglycoside | Trospectomycin | | ? | | | | | J Chemother 1995 Dcc; |
| | | | | | | | | 7(6): 515-8 |
| 4,5-disubstituted | Isepamycin | | | | | | | |
| deoxystreptamine | | | | | | | | |
| core | | | | | | | | |
| 4,5-disubstituted | Kanamycin C | | | | | | | |
| deoxystreptamine | | | | | | | | |
| core | | | | | | | | |
| 4,5-disubstituted | Arbekacin | | | | | | | |
| deoxystreptamine | | | | | | | | |
| core | | | | | | | | |

The following aminoglycosides have an amine group which can be labelled with a fluorophore:

| Class | Antibiotic | Target | RRNA | Nucleotides(s) | Ref |
|---|---|---|---|---|---|
| Aminoglycoside | Amikacin | A site | 16S | 1408 | 11 |
| Aminoglycoside | Apramycin | A site | 16S | 1408 1419 1494 | 12 |
| Aminoglycoside | Bekanamycin | site | 16S | ? | 13 |
| Aminoglycoside | Gentamicin | A site (L6) | 16S | 1408 1419 1494 | 14 |
| Aminoglycoside | Hygromycin B | | 16S | 1491 1495 | 15 |
| Aminoglycoside | Kanamycin | A site | 16S | 1408 1419 1494 | 16 |
| Aminoglycoside | Kasugamycin | | 163 | 794 926 | 12 |
| Aminoglycoside | Neamin | A site | 16S | 1408 1419 1494 | 12 |
| Aminoglycoside | Neomycin | A site | 16S | 1408 1419 1494 | 17 |
| Aminoglycoside | Paromomycin | A site | 16S | 1408 1419 1491 1494 | 18 |
| Aminoglycoside | Sisomycin | | | | 19 |
| Aminoglycoside | Spectinomycin | S5 binding site | 16S | 1063-1065 1191-1193 | 20 |
| Aminoglycoside | Tobramycin | A site | 163 | ? | 21 |

A number of reactions can be used to label amines, including but not limited to the following:

| Reaction | Product |
|---|---|
| dye-isothiocyanates | Thiourea |
| dye-succinimidyl ester | Carboxamide |
| dye-sulfonyl chloride | Sulphonamide |
| dye-aldehyde | Alkylamine |

Streptomycin contains an aldehyde group that is appropriate for the introduction of fluorescent dyes:

| Class | Antibiotic | Target | rRNA | Nucleotides(s) | ref |
|---|---|---|---|---|---|
| Amino- | Streptomycin | A site, 915 | 16S | 523 911-915 | 22 |
| glycoside | | region, S12, | | | |
| | | S5?, L11? | | | |

A number of reactions can be used to label aldehydes, including but not limited to the following:

| Reaction | Product |
|---|---|
| dye-hydrazides | Hydrazones |
| dye-semicarbazides | Hydrazones |
| dye-carbohydrazides | Hydrazones |
| dye-amines | Alkylamine |

Preferred aminoglycosides are those which interact at the ribosome A site. Preferred aminoglycosides for use according to the invention are 4,5-disubstituted deoxystreptamines, such as neomycin and paromomycin, in particular paromomycin. A further preferred aminoglycoside is streptomycin. A further group of preferred aminoglycosides are 4,6 deoxystreptamines, such as tobramycin and gentamycin.

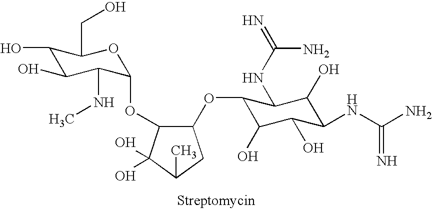

| | R₁ |
|---|---|
| Neomycin | NH₂ |
| Paramomycin | OH |

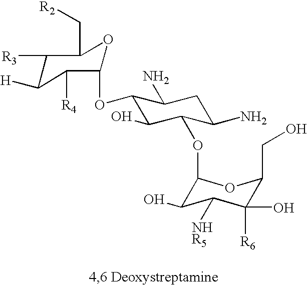

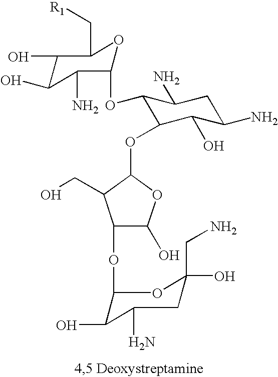

| | R₂ | R₃ | R₄ | R₅ | R₆ |
|---|---|---|---|---|---|
| Tobramycin | NH₂ | OH | OH | H | H |
| Gentamycin | NHCH₃ | H | NH₂ | CH₃ | CH₃ |

It is surprising that fluorescent labels can be attached to RNA-binding compounds without affecting the ability of these compounds to bind to large RNA molecules because (a) the chemical groups to which labels can be attached are generally the same as those which interact with RNA and (b) even though it may be relatively large (e.g. doubling the size of the initial molecule), the fluorescent label does not interfere with the binding interaction. The reporter will not typically possess intrinsic fluorescence in its unlabelled form.

In contrast to the methods disclosed in reference 1, the fluorescent group on the reporter does not interact with a fluorescent group on the RNA target. The alignment of fluorescent groups in reference 1 implies that the site in the RNA which interacts with the reporter is known whereas, in the present invention, this is not necessary; where this information is known, however, the label will typically be attached at a site remote from the main interacting groups.

It will be appreciated that the methods of the present invention are not aimed at revealing sites within RNA which are bound by the reporter. Conversely, however, they may reveal the sites within the reporter which bind to the RNA.

The Test Compound

The method of the invention may be used to identify compounds capable of binding to any large RNA target, preferably as part of a screening process.

Typical test compounds include, but are not restricted to, peptides, peptoids, proteins, lipids, metals, nucleotides, nucleosides, small organic molecules, antibiotics, polyamines, and combinations and derivatives thereof. Small organic molecules have a molecular weight of more than 50 and less than about 2,500 daltons, and most preferably between about 300 and about 800 daltons. Complex mixtures of substances, such as extracts containing natural products, or the products of mixed combinatorial syntheses, can also be tested and the component that binds to the target RNA can be purified from the mixture in a subsequent step.

Test compounds may be derived from large libraries of synthetic or natural compounds. For instance, synthetic compound libraries are commercially available from Maybridge Chemical Co. (Trevillet, Cornwall, UK) or Aldrich (Milwaukee, Wis.). Alternatively, libraries of natural compounds in the form of bacterial, fungal, plant and animal extracts may be used. Additionally, test compounds may be synthetically produced using combinatorial chemistry either as individual compounds or as mixtures.

Test compounds which can displace reporters based on known antibiotics are useful lead compounds for the development of improved antibiotics.

Indicator Molecules

Contact between the pair of indicator molecules may occur in solution (e.g., a test tube, dish or well of a microtitre plate) or, alternatively, either the reporter molecule or the RNA target molecule may be adhered to a solid support (e.g. an affinity gel, matrix, or column) by covalent or non-covalent linkages using methods known in the art. The support bound RNA target or reporter molecule is then mixed with a solution containing the other compound of the indicator pair.

In some embodiments, a fraction of the reporter molecules and RNA target molecules in the binding reaction can be replaced by unlabelled analogues. The optimal proportions of labelled and unlabelled reporting and RNA target molecules can be determined by titration of the different components and measuring the optimal concentrations.

The RNA and labelled reporter molecules are then mixed with a test compound and the fluorescence in the mixture is measured. If the test compound is able to bind to the region of the target RNA that binds to the reporter, then a fraction of the reporter will be prevented from binding to the RNA target. The proportions of the free reporter, free RNA and complex can be quantitatively determined by comparing the special properties of the complex, partially dissociated complex and the uncomplexed target RNA and reporters. The amount of reporter displacement will be a function of the relative affinity of the test compound for the RNA target compared to the reporter and the relative concentrations of the two molecules in the sample. Preferably, a variety of different concentrations of the molecule to-be-tested are compared to generate a binding curve.

The concentration of compounds binding to RNA targets can be determined with a fluorescence standard curve depicting the fluorescence of the labelled reporter and RNA targets with varying known concentrations of competing unlabelled test compound.

In some embodiments of the invention, the test compound is first mixed with the RNA in order to form a complex in the absence of the labelled reporter, and the reporter is then added. Since the reporter will only be able to bind to the free RNA in the reaction, there will be a reduced amount of complex formed between the reporter and the RNA target compared to the amount of complex formed in the absence of test compound.

In other embodiments, a complex is pre-formed between the RNA and the labelled reporter before addition of the test compound. If the test compound is able to disrupt the complex formed between the RNA and the labelled reporter, or alter the equilibrium binding state by binding to RNA that has dissociated from the reporter, the amount of complex in the reaction will be reduced.

Measurable Changes

The displacement of the fluorescent reporter from the large RNA target can be measured by a number of techniques, including:

fluorescence anisotropy [(I_{//}−I⊥)/(I_{//}+2I⊥), where I_{//} is the fluorescence intensity viewed through parallel polarisers and I⊥ is the fluorescence intensity viewed through orthogonal polarisers]. This is a particularly useful method when dealing with large RNA targets. In solution, a reporter is generally rotationally free with low anisotropy. When bound to the RNA target, however, they become rotationally constrained and anisotropy is high. Displacement of the reporter is thus associated with a reduction in anisotropy.

fluorescence polarisation [(I_{//}−I⊥)/(I_{//}+I⊥)]

a change in fluorescence intensity or quenching occurring on dissociation. The intensity of many fluorophores depends on the local environment. The change from free solution to the environment of the large RNA target may thus result in a change in fluorescence intensity and/or quenching.

The method includes the step of comparing the fluorescence of reporter molecule in the presence and absence of the test compound. It will be appreciated that the fluorescence of the reporter in the absence of the test compound may have been determined before performing the method, or may be determined during or after the method has been performed. It may be an absolute standard.

Library Screening (Including High Throughput Screens)

The present invention also encompasses high-throughput screening methods for identifying compounds that bind to a RNA target. Preferably, all the biochemical steps for this assay are performed in a single solution in, for instance, a test tube or microtitre plate, and the test compounds are analyzed initially at a single compound concentration. For the purposes of high throughput screening, the experimental conditions are adjusted to achieve a proportion of test compounds identified as “positive” compounds from amongst the total compounds screened. The assay is preferably set to identify compounds with an appreciable affinity towards the RNA target e.g., when 0.1% to 1% of the total test compounds from a large compound library are shown to bind to a given RNA target with a Ki of 10 μM or less (e.g. 1 μM, 100 nM, 10 nM, or less).

Kits Useful According to the Invention

The invention also provides a kit for determining whether a text compound binds to a large RNA target, the kit comprising (i) a large RNA target; and (ii) a fluorescent reporter molecule, wherein the reporter molecule is an oxazolidinone or an aminoglycoside.

Measurements of RNA Binding Compound

The invention may be embodied as a clinical assay or method for determining the presence of an RNA-binding compound in a biological sample such as the serum or tissues of a subject. Many drugs, including RNA-binding compounds such as antibiotics, are routinely assayed for their serum levels when administered to patients to prevent administration of toxic levels of compounds.

The invention thus provides a method for determining the presence in a biological sample of a compound that binds to a large RNA target, the method comprising the steps of (a) contacting the sample with a pair of indicator molecules comprising (i) the large RNA target; and (ii) a fluorescent reporter molecules; wherein the reporter molecule is an oxazolidinone or an aminoglycoside; and (b) measuring the fluorescence of the reporter molecule in the presence of the sample and comparing this value to the fluorescence of the reporter in the absence of the test compound.

The invention also provides a kit for determining the level of an RNA-binding compound of interest in a subject or sample, comprising (i) a large RNA target; and (ii) a fluorescent reporter molecule wherein the reporter molecule is an oxazolidinone or an aminoglycoside.

### BRIEF DESCRIPTION OF DRAWINGS

FIG. 1 shows the structure of TAMRA-labelled oxazolidinone.

FIG. 2 shows the results of a ribosome binding assay using TAMRA-labelled oxazolidinone, with the circles (∘) showing % binding.

FIG. 3 shows the site and structure of the E-site RNA within the rRNA, and FIG. 4 shows the titration of TAMRA-labelled oxazolidinone against the E-site. FIG. 5 shows similar data for binding to rRNA.

FIG. 6 shows the competitive binding data for labelled (∘) and non-labelled (●) oxazolidinone.

FIG. 7 shows data from a similar competitive binding experiment using labelled oxazolidinone (∘) and linezolid (●).

FIG. 8 shows the results of a ribosome binding assay using TAMRA-labelled paromomycin.

### MODES FOR CARRYING OUT TILE INVENTION

### EXAMPLE 1

Preparation of Oxazolidinone Reporter

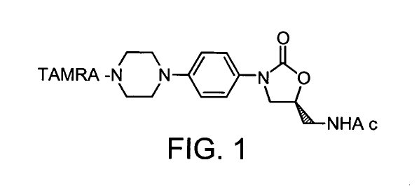

TAMRA-labelled oxazolidinone (FIG. 1) was synthesised by reacting 4 mg oxazolidinone hydrochloride in sodium bicarbonate (6 mL 0.067M in 30% dimethyl formamide (DMF)) with 5 mg 5-carboxytetramethyl rhodamine (in 1 mL DMF) over 12 hours at room temp. The solution was diluted and purified by anion exchange chromatography and reverse phase HPLC.

### EXAMPLE 2

Binding Assay with Oxazolidinone Reporter and Ribosomes

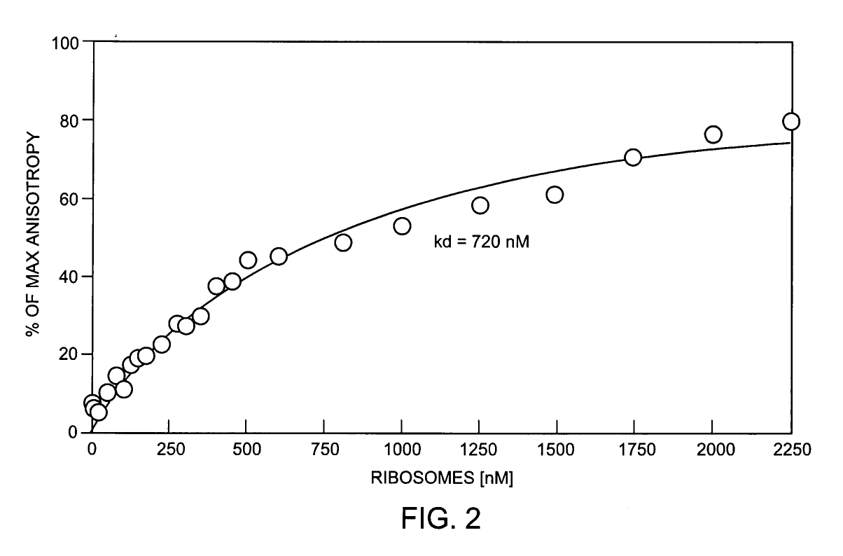

The interaction between oxazolidinone-TAMRA and E. coli ribosomes was monitored by measuring the change in fluorescence anisotropy. Each measurement was made in a 400 μL cuvette, in a Perkin Elmer LS50B fluorimeter. Increasing amounts of ribosomes (corresponding to the amounts shown in FIG. 2) were added to a solution of 50 nM oxazolidinone-TAMRA in the presence of 50 mM Tris.HCl pH 7.5, 70 mM NH₄Cl, 30 mM KCl, 7 mM MgCl₂, 1 mM DTT, 0.5 mM EDTA. For each titration point an anisotropy measurement was acquired using an excitation wavelength of 554 nm with the excitation slits set to 10 nm and an emission wavelength of 575 nm with the emission slits set to 10 nm. The values presented in FIG. 2 are the average of three fluorescence anisotropy measurements expressed as a percentage of the maximum anisotropy value.

The anisotropy shows an increase upon addition of the ribosomes. This is consistent with binding of the TAMRA-oxazolidinone to the ribosome, with a dissociation constant of 720 nM.

### EXAMPLE 3

Binding Assay with Oxazolidinone Reporter and E-Site RNA

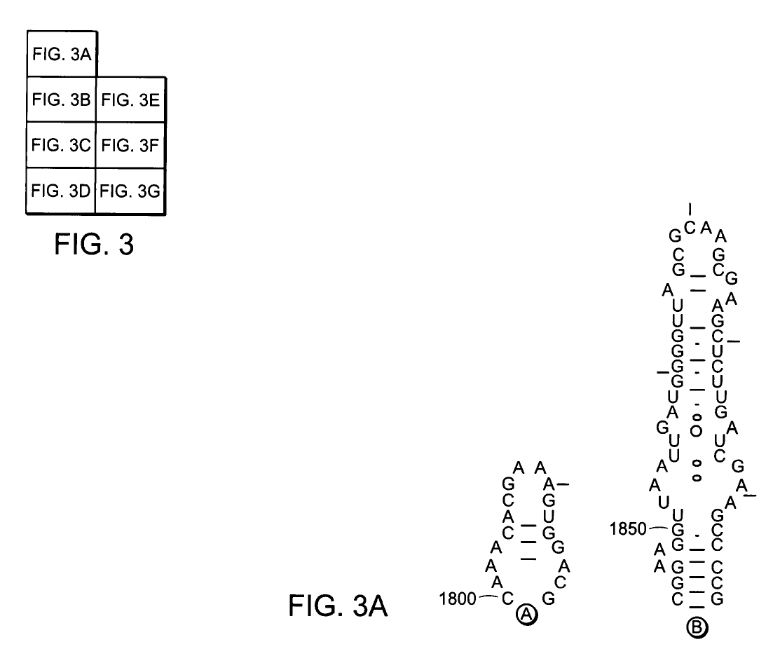

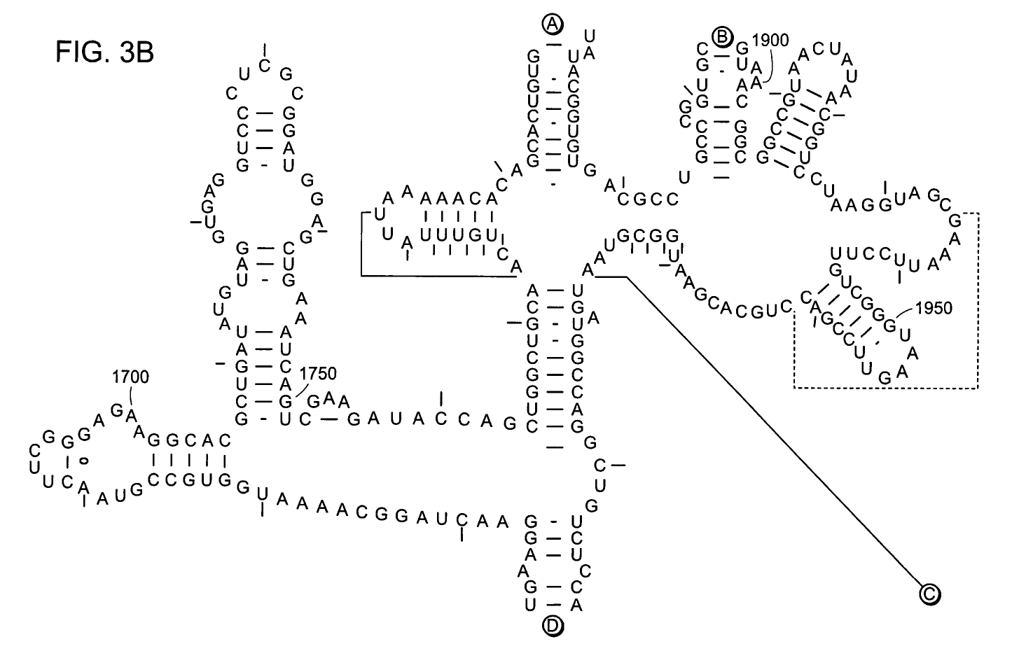

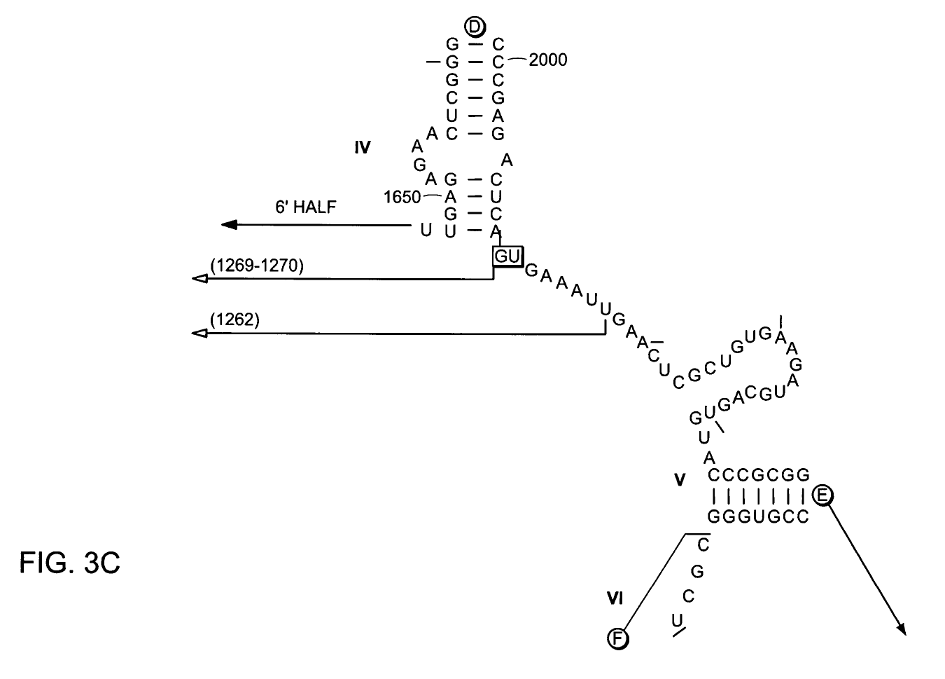

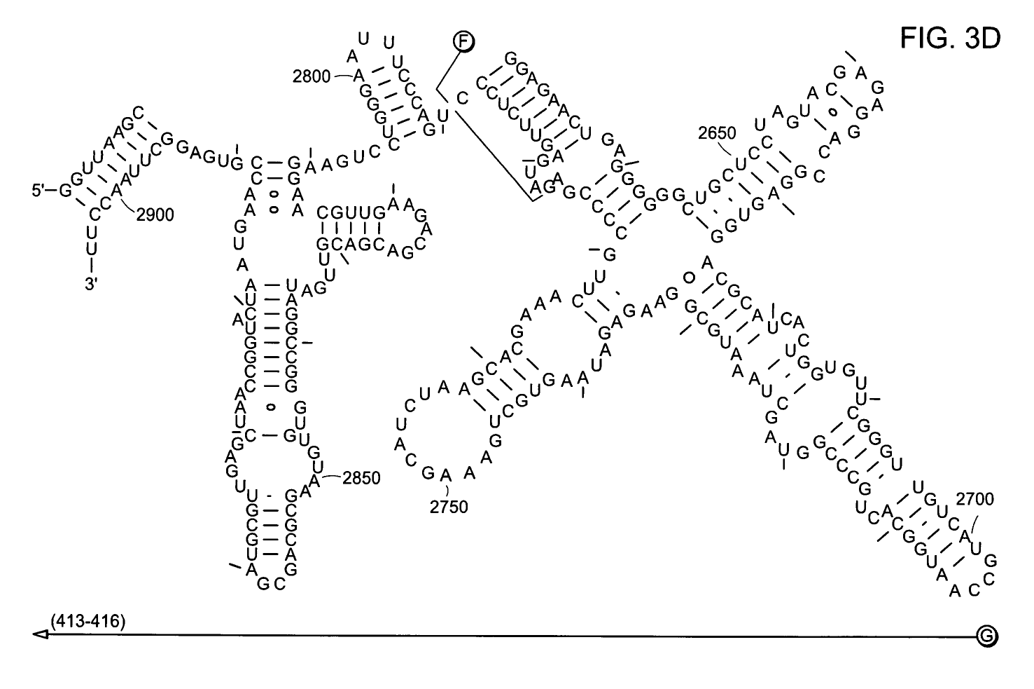

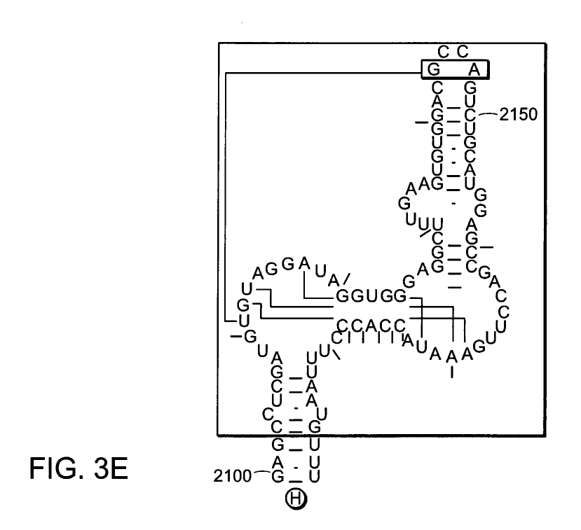

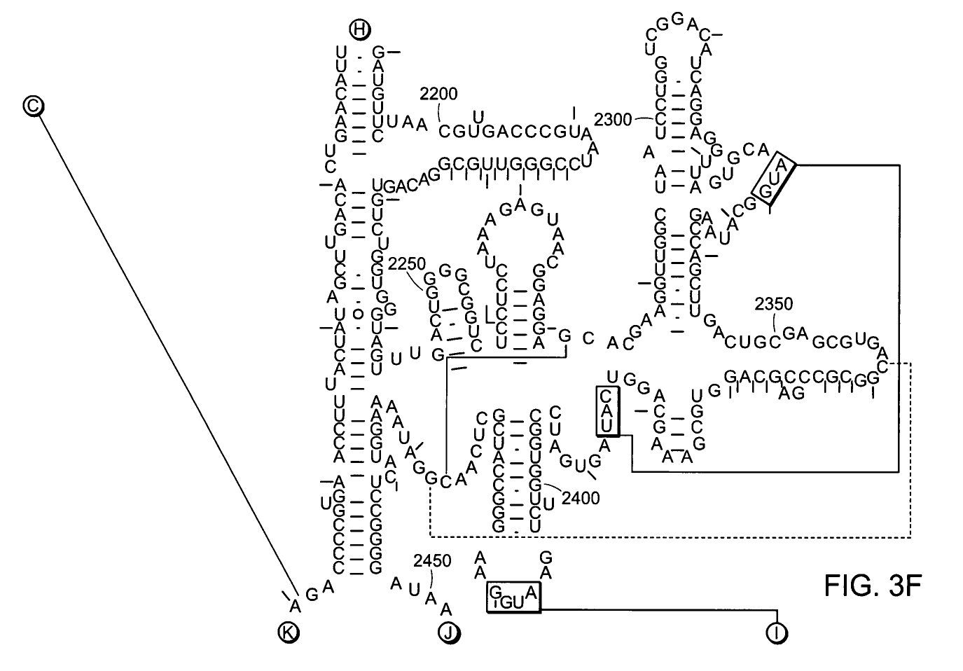

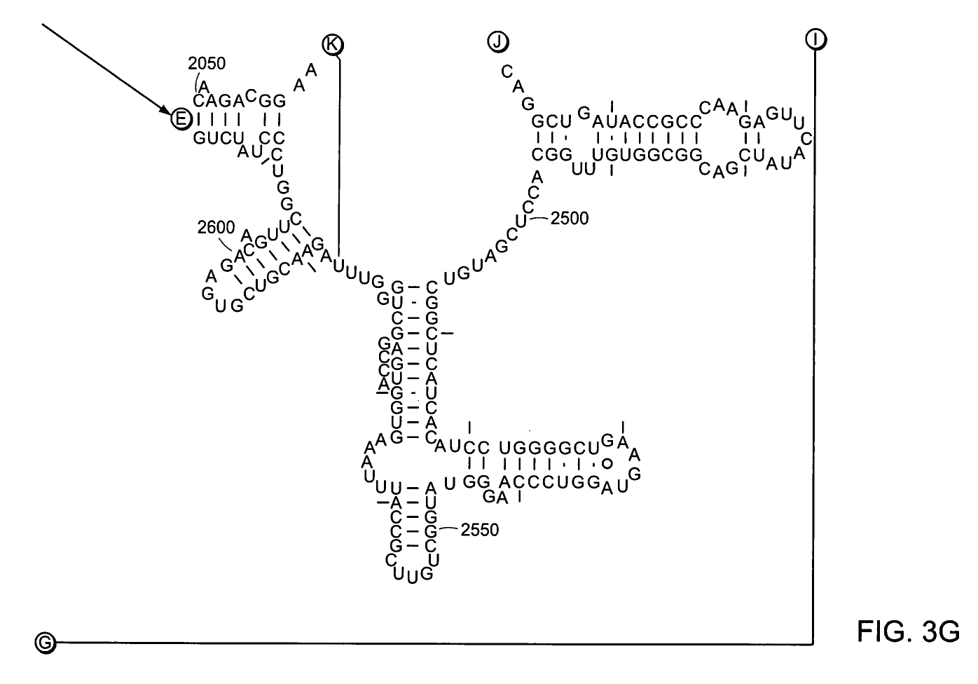

The interaction between oxazolidinone-TAMRA and a small E. coli rRNA sequence that has been identified as the binding site for the oxazolidinones (E-site RNA—FIG. 3) was monitored by measuring the change in fluorescence anisotropy. Each measurement was made in a 400 μL cuvette, in a Perkin Elmer LS50B fluorimeter, increasing amounts of E site RNA, an 82 nucleotide oligoribonucleotide (corresponding to the amounts shown in FIG. 4) were added to a solution of 50 nM oxazolidinone-TAMRA in the presence of 50 mM Tris.HCl pH7.5, 70 mM NH₄Cl, 30 mM KCl, 7 mM MgCl₂, 1 mM DTT, 0.5 mM EDTA. For each titration point an anisotropy measurement was acquired using an excitation wavelength of 554 nm with the excitation slits set to 10 nm and an emission wavelength of 575 nm with the emission slits set to 10 nm. The values presented were the average of three fluorescence anisotropy measurements expressed as a percentage of the maximum anisotropy value.

The anisotropy shows no increase upon addition of the E-site RNA. This is consistent with non-recognition of the small ‘minimal’ E-site RNA by oxazolidinone-TAMRA. Thus the trend in references 1 and 2 towards using small RNA molecules for binding studies is not suitable for studying the interaction of oxazolidinones with ribosomes—whereas oxazolidinones bind to the whole ribosome at the E-site, they do not bind to the E-site in isolation.

### EXAMPLE 4

Binding Assay with Oxazolidinone Reporter and rRNA

The interaction between oxazolidinone-TAMRA and E. coli ribosomal RNA was monitored by measuring the change in fluorescence anisotropy. Each measurement was made in a 400 μL cuvette, in a Perkin Elmer LS50B fluorimeter, increasing amounts of ribosomal RNA (corresponding to the amounts shown in FIG. 5) were added to a solution of 50 nM oxazolidinone-TAMRA in the presence of 50 mM Tris.HCl pH7.5, 70 mM NH₄Cl, 30 mM KCl, 7 mM MgCl₂, 1 mM DT, 0.5 mM EDTA. For each titration point an anisotropy measurement was acquired using an excitation wavelength of 554 nm with the excitation slits set to 5 nm and an emission wavelength of 575 nm with the emission slits set to 10 nm. The fluorescence anisotropy values presented were the average of three measurements.

In contrast to the minimal ‘E-site’ RNA, the measured anisotropy shows an increase in fluorescence anisotropy upon addition of the ribosomal RNA. This is consistent with binding of the oxazolidinone-TAMRA to the ribosomal RNA, with a dissociation constant of 1600 nM. Thus a small RNA is unsuitable for studying the interaction of oxazolidinones with ribosomes, but the complete rRNA can be used.

### EXAMPLE 5

Competition Assays with Oxazolidinone Reporter and Ribosomes

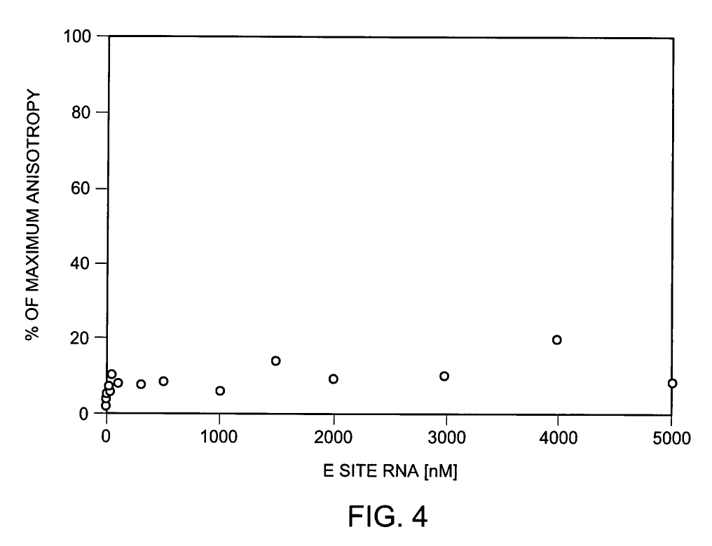

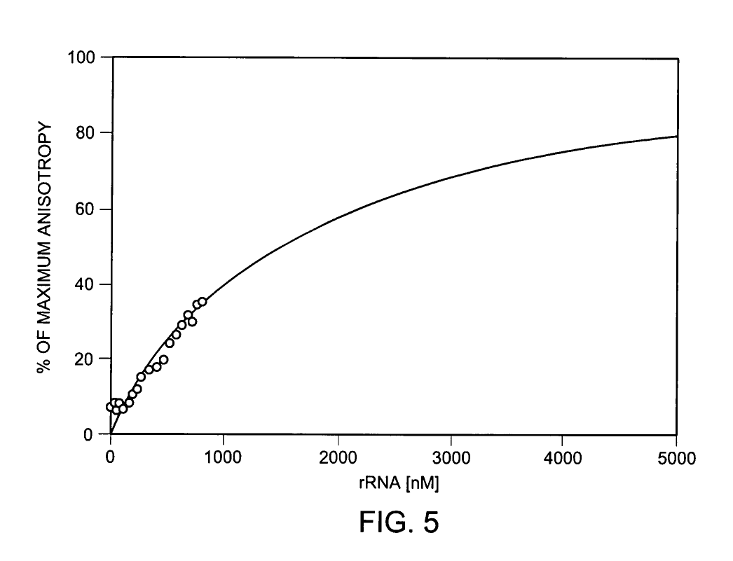

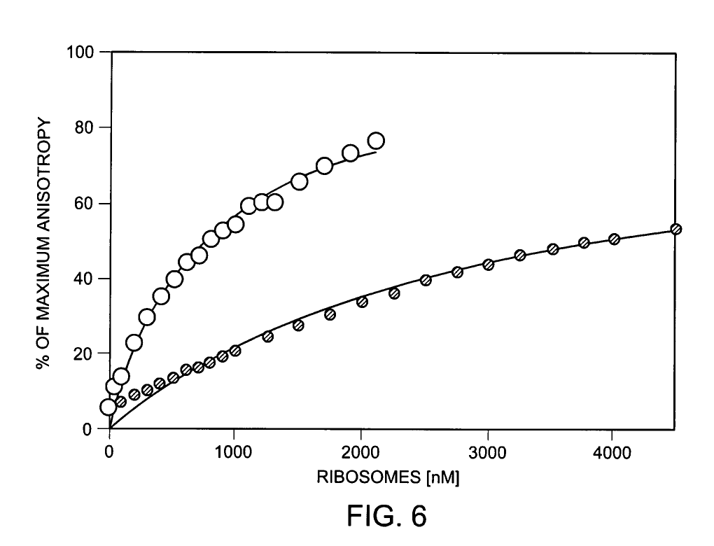

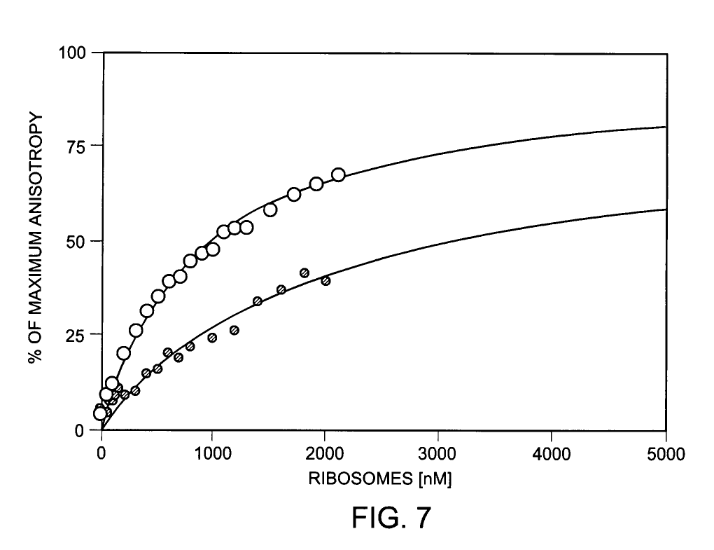

The interaction between oxazolidinone-TAMRA and E. coli ribosomes was monitored by comparing the change in fluorescence anisotropy in the presence and absence of 100 μM non-fluorescent oxazolidinone competitor. Each measurement was made in a 400 μl cuvette, in a Perkin Elmer LS50B fluorimeter. Increasing amounts of ribosomes (corresponding to the amounts shown in FIG. 6) were added to a solution of 20 nM oxazolidinone-TAMRA in the presence of 50 mM Tris.HCl pH 7.5, 70 mM NH₄Cl, 30 mM KCl, 7 mM MgCl₂, 1 mM DTT, 0.5 mM EDTA. For each titration point an anisotropy measurement was acquired using an excitation wavelength of 554 nm with the excitation slits set to 10 nm and an emission wavelength of 575 nm with the emission slits set to 10 nm. The values presented in FIG. 6 are the average of three fluorescence anisotropy measurements expressed as a percentage of the maximum anisotropy.

In the presence of non-fluorescent oxazolidinone competitor, the anisotropy is considerably reduced upon addition of the ribosomes. This is consistent with inhibition of the binding of TAMRA-oxazolidinone to the ribosome by the non-fluorescent oxazolidinone competitor.

The same experiment was performed, but using 20 nM non-fluorescent linezolid (an oxazolidinone) as the competitor. In the presence of the competitor the anisotropy is considerably reduced upon addition of the ribosomes. This is consistent with inhibition of the binding of TAMRA-oxazolidinone to the ribosome by linezolid.

### EXAMPLE 6

Preparation of Paromomycin Reporter

TAMRA-labelled paromomycin was synthesised by reacting 55 mg paromomycin sulphate in sodium bicarbonate (6 mL 0.067M in. 30% DMF) with 5 mg 5-carboxytetramethyl rhodamine (in 1 mL DMF) over 12 hours at room temp. The solution was diluted and purified by anion exchange chromatography, and reverse phase HPLC [23].

### EXAMPLE 7

Binding Assay with Paromomycin Reporter and Ribosomes

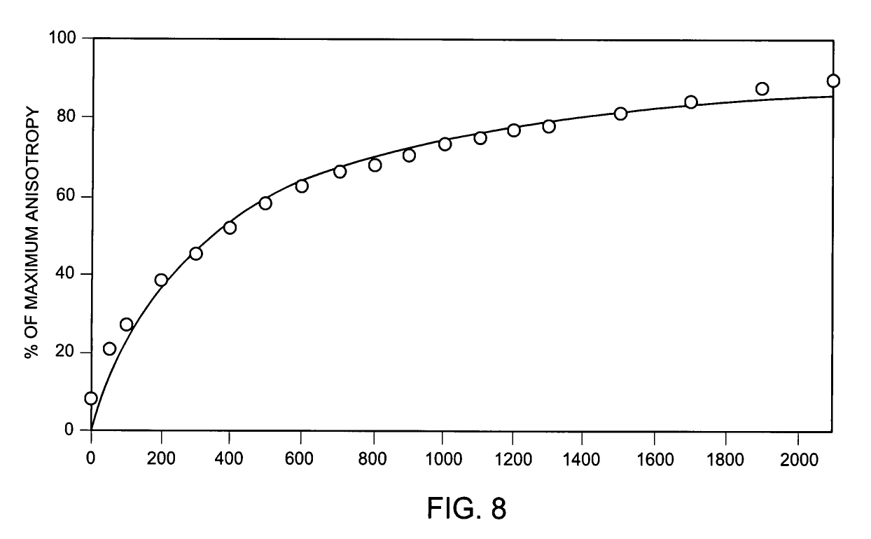

The interaction between paromomycin-TAMRA and E. coli ribosomes was monitored by measuring the change in fluorescence anisotropy. Each measurement was made in a 400 μL cuvette, in a Perkin Elmer LS50B fluorimeter. Increasing amounts of ribosomes (corresponding to the amounts shown in FIG. 8) were added to a solution of 50 nM paromomycin-TAMRA in the presence of 50 mM Tris.HCl pH 7.5, 70 mM NH₄Cl, 30 mM KCl, 7 mM MgCl₂, 1 nM DTT, 0.5 mM EDTA. For each titration point an anisotropy measurement was acquired using an excitation wavelength of 554 nm with the excitation slits set to 5 nm and an emission wavelength of 575 nm with the emission slits set to 10 nm. The fluorescence anisotropy values in FIG. 8 presented are the average of three measurements.

The measured anisotropy shows an increase in fluorescence anisotropy upon addition of the ribosomes. This is consistent with binding of the TAMRA-paromomycin to the ribosome, with a dissociation constant of 330 nM.

It will be understood that the invention has been described by way of example only and modifications may be made whilst remaining within the scope and spirit of the invention.

References

All references are incorporated herein by reference.

[1] WO99164625 (RiboTargets)

[2] U.S. Pat. No. 5,593,835

[3] Matassova et al. (1999) RNA 5:939-946

[4] Brickner et al., (1996) J. Med Chem. 39:673-79

[5] Tucker et al. (1998) J. Med. Chem. 41:3727-3735

[6] Barbachyn et al. Bioorganic & Medicinal Chemistry Letters 6(9): 1003-1008

[7] Barbachyn et al. Bioorganic & Medicinal Chemistry Letters 6(9): 1009-1014

[8] Pae et al. (1999) Bioorganic & Medicinal Chemistry Letters 9:2679-2684

[9] Pae et al. (1999) Bioorganic & Medicinal Chemistry Letters 9:2685-2690

[10] Rheinberger et al. (1981) PNAS USA 78:53 10-14

[11] J Infect Dis 1998 Jun;177(6):1573-81

[12] EMBO J 1991 Oct;10(10):3099-103

[13] Biochemistry 1998 Jan 13;37(2):656-63

[14] EMBO J 1998 Nov 16;17(22):6437-48

[15] Biochemistry 1984 Mar 27;23(7):1462-7

[16] J Mol Biol 1987 Feb 20;193(4):661-71

[17] J Mol Biol 1999 Feb 12;286(1):33-43

[18] J Mol Biol. 1998 Mar 27;277(2):333-45

[19] J Bacteriol. 1992 Dec; 174(23):7868-72.

[20] J Mol Biol. 1997 Aug 29;271(4):566-87

[21] Nature 1987, 327, 389-394

[22] J Mol Biol. 1997 Oct 31;273(3):586-99.

[23] Biochemistry 1997 36:768-779

[25] Riedl & Endermann, Exp. Opin. Ther. Patents (1999), 2(5), 625-633

[26] Genin, Exp. Opin. Ther. Patents (2000), 10(1), 1405-1414

1. A method for determining whether a test compound binds to a large RNA target, the method comprising the steps of:
(a) contacting the test compound with a pair of indicator molecules comprising (i) the large RNA target; and (ii) a fluorescent reporter molecule, wherein the reporter molecule is an oxazolidinone or an aminoglycoside; and
(b) measuring the fluorescence of reporter molecule in the presence of the test compound and comparing this value to the fluorescence of the reporter in the absence of the test compound.

2. The method of claim 1, wherein the large RNA target comprises 250 nucleotides or more.

3. The method of claim 1, wherein the large RNA target comprises 500 nucleotides or more.

4. The method of claim 1, wherein the large RNA target contains at least 0.5N base pairs,
wherein N=0.5(total number of nucleotides in the large RNA target).

5. The method of claim 1, wherein the large RNA target comprises at least one of the following secondary structure motifs: bulged (non base-paired) and extra-helical bases; internal loops; helical junctions; G-quartets; and pseudoknots.

6. The method of claim 1, wherein the large RNA target is a viral genome, a RNA comprising an IRES, a nucleoprotein complexes, a mRNA, a rRNA, a ribosomal subunit, or a whole ribosome.

7. The method of claim 1, wherein the fluorescent reporter molecule is labelled with TAMRA.

8. The method of claim 1, wherein large RNA target comprises the E-site RNA.

9. The method of claim 1, wherein step (b) involves the measurement of fluorescence anisotropy, fluorescence polarization, and/or a change in fluorescence intensity or quenching.

10. A fluorescently-labelled oxazolidinone.

11. The labelled oxazolidinone of claim 10, wherein the label is attached to an aldehyde group of the oxazolidinone.

12. The labelled oxazolidinone of claim 10 or claim 11, wherein the label is TAMRA.

13. The labelled oxazolidinone of claim 12, as shown in FIG. 1.

14. A kit for determining whether a test compound binds to a large RNA target, the kit comprising (i) a large RNA target; and (ii) a fluorescent reporter molecule, wherein the reporter molecule is an oxazolidinone or an aminoglycoside.

15. A method for determining the presence in a biological sample of a compound that binds to a large RNA target, the method comprising the steps of (a) contacting the sample with a pair of indicator molecules comprising (i) the large RNA target; and (ii) a fluorescent reporter molecule; wherein the reporter molecule is an oxazolidinone or an aminoglycoside; and (b) measuring the fluorescence of the reporter molecule in the presence of the sample and comparing this value to the fluorescence of the reporter in the absence of the test compound.


SEQUENCE LISTING (1 sequence)

SEQ ID NO: 1 - RNA, 1258 nt, Escherichia coli
uugagagaacucgggugaaggaacuaggcaaaauggugccguaacuucgggagaaggcacgcugauauguaggugaggucccucgcggauggagcugaaaucagucgaagauaccagcuggcugcaacuguuuauuaaaaacacagcacugugcaaacacgaaaguggacguauacggugugacgccugcccggugccggaagguuaauugaugggguuagcgcaagcgaagcucuugaucgaagccccgguaaacggcggccguaacuauaacgguccuaagguagcgaaauuccuugucggguaaguuccgaccugcacgaauggcguaaugauggccaggcugucuccacccgagacucagugaaauugaacucgcugugaagaugcaguguacccgcggcaagacggaaagaccccgugaaccuuuacuauagcuugacacugaacauugagccuugauguguaggauaggugggaggcuuugaaguguggacgccagucugcauggagccgaccuugaaauaccacccuuuaauguuugauguucuaacguugacccguaauccggguugcggacagugucugguggguaguuugacuggggcggucuccuccuaaagaguaacggaggagcacgaagguuggcuaauccuggucggacaucaggagguuagugcaauggcauaagccagcuugacugcgagcgugacggcgcgagcaggugcgaaagcaggucauagugauccggugguucugaauggaagggccaucgcucaacggauaaaagguacuccggggauaacaggcugauaccgcccaagaguucauaucgacggcgguguuuggcaccucgaugucggcucaucacauccuggggcugaaguaggucccaaggguauggcuguucgccauuuaaagugguacgcgagcuggguuuagaacgucgugagacaguucggucccuaucugccgugggcgcuggagaacugaggggggcugcuccuaguacgagaggaccggaguggacgcaucacugguguucggguugucaugccaauggcacugcccgguagcuaaaugcggaagagauaagugcugaaagcaucuaagcacgaaacuugccccgagaugaguucucccugacccuuuaaggguccugaaggaacguugaagacgacgacguugauaggccggguguguaagcgcagcgaugcguugagcuaaccgguacuaaugaaccgugaggcuuaaccuu